(19) United States
(12) Patent Application Publication
(10) Pub. No.: US 2025/0107551 A1
(43) Pub. Date: Apr. 3, 2025

(54) DECOLORIZED SPIRULINA AND METHODS FOR PRODUCING THE SAME

(71) Applicant: ALGAECORE TECHNOLOGIES LTD., Kibutz Glil-Yam (IL)

(72) Inventors: Ilya PAKHOMOV, Rishon LeZion (IL); Baruch DACH, Tel-Mond (IL); Lior SHAUDER, Kibbutz Lahav (IL)

(73) Assignee: ALGAECORE TECHNOLOGIES LTD., Kibutz Glil-Yam (IL)

(21) Appl. No.: 18/840,016

(22) PCT Filed: Feb. 21, 2023
(86) PCT No.: PCT/IL2023/050183

§ 371 Date: Aug. 20, 2024

Related U.S. Application Data
(60) Provisional application No. 63/268,443, filed on Feb. 24, 2022.

(51) Int. Cl. A23J 1/00; A23L 17/60; B33Y 80/00; C12N 1/12; C12R 1/89

(52) U.S. Cl. CPC A23J 1/008; A23L 17/60; B33Y 80/00; C12N 1/12; C12R 2001/89

(57) ABSTRACT

Spirulina biomass and food products from spirulina biomass having color characteristics that enable additional applications in food industry are provided.

### FIELD OF INVENTION

The present invention relates to the field of spirulina-based products, specifically spirulina-based products having a specific color and beneficial properties.

### BACKGROUND OF THE INVENTION

Spirulina has long been known for its superior properties. Due to its non-animal origin and excellent nutritional value, spirulina is frequently referred as a super-food. As such, it is widely used by both consumers and food manufactures, and becomes more and more popular as the demand for vegetarian and vegan products increases. Spirulina-based products are characterized by strong green color attributed to phycobiliproteins, specifically, phycocyanin. While for certain applications the color is not a downside, or even a beneficial property, it is a serious disadvantage for the segment of meat, fish, and dairy alternatives. It stems from the fact that in addition to the nutritional value and taste, the appearance of the product, its color and texture should resemble the original product (whether its meet, fish and/or cheese). Green cheese or steak is unlikely to become a valid alternative even though it contains 100% of high-quality protein of a non-animal source.

As providing a valid alternative for the food industry in general, and specifically for the segment of meat, fish, and dairy alternatives remains a long and unmet need, Spirulina-based products without green pigment can be tremendously beneficial.

### SUMMARY OF THE INVENTION

It is a principal object of the invention to provide spirulina biomass and food products from spirulina biomass having color characteristics that enable additional applications in food industry.

According to some embodiments, the invention provides food-grade spirulina biomass characterized by color coordinates in the range of 15<L<90, 0<a<95, 0<b<55, wherein the maximum value (a+b) is equal to or smaller than (L*1.2), as measured according to CIELAB color space.

According to some embodiments, the invention provides a food-grade spirulina biomass, characterized by having spectrum absorbance at the maximum absorbance peaks in the range of 400-560 nanometers.

According to some embodiments, the invention provides a food-grade spirulina biomass characterized by RGB color space values in the range of 50>R>255 wherein each G and B is equal to or smaller than (R−30).

According to some embodiments, the invention provides a food-grade spirulina biomass characterized by CMYK color space values of C=0, 0.1>M>0.8, 0.1>Y>0.8, 0.01>K>0.5, wherein K<M*0.6 and M*1.5>Y>M*0.5.

According to some embodiments, the invention provides an edible media prepared from and/or comprising the food-grade biomass according to the above embodiments.

According to some embodiments, the invention provides a food-grade bio-ink prepared from and/or comprising the biomass or the edible media according to the above embodiments.

In one embodiment, the invention provides a 3D printed food product prepared from the bio-ink according to the above embodiments.

According to some embodiments, the invention provides a food product prepared from and/or comprising the biomass or the edible media according to the above embodiments.

According to some embodiments, the invention provides a process for the preparation of spirulina-based biomass characterized by color coordinates in the range of 15<L<90, 0<a<95, 0<b<55, wherein the maximum value of (a+b) is equal to or smaller than (L*1.2), as measured according to CIELAB color space comprising the steps of:
a. mixing Spirulina biomass with an organic solvent;
b. adding to the mixture an alkaline component to reach pH in the range of 8.0 to 14.0;
c. adding to the mixture a peroxide solution;
d. Homogenizing the mixture;
e. optionally, cooling the resulted mixture to the room temperature;
f. separating the biomass from the medium;
g. washing the biomass with an aqueous solution comprising a fresh polar organic solvent;
h. suspending the washed biomass in the water and reducing the pH to the range of 3.0 to 4.0; and,
i. separating the biomass and resuspending in water.

### BRIEF DESCRIPTION OF THE DRAWINGS

FIG. 1A-C represents L, a, b, C, h values for food and Spirulina samples;

FIG. 2A-F represents distribution of spirulina biomass having the following shades: 2A orange; 2B dark orange; 2C pink; 2D white; 2E salmon; 2F red; and,

FIG. 3 represents protein content measurements of the spirulina samples.

### DETAILED DESCRIPTION OF THE INVENTION

The present invention is now described more fully hereinafter with reference to the accompanying examples and drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

According to some embodiments, the invention provides a food-grade spirulina biomass characterized by color coordinates in the range of 15<L<90, 0<a<95, 0<b<55, wherein the maximum value (a+b) is equal to or smaller than (L*1.2), as measured according to CIELAB color space.

CIELAB is used for quantifying the shades of products. The scale principle is based on 3 axes. Axis, L* that quantifies the whiteness or darkness of the product, where 0 is dark “black” and 100 is light or “white”. In the axis, a*—red (100 (+to green (100−)). On the b* axis is yellow (100+) to blue (100−). Each color—hue—can be quantified by determining the above three values. In addition to L*b*a* values, there are a number of indexes for characterizing specific colors and effects. The root of the differential sum of L*a*b* squared is (dE)

$$Δ E = \sqrt{{( Δ {L}^{⋆} )}^{2} + {( Δ {a}^{⋆} )}^{2} + {( Δ {b}^{⋆} )}^{2}}$$

which value expresses the difference of all color values in one number. It is accepted that a value above 1.5 to 2.0 about dE* is the limit for distinguishing the eye.

In the context of the invention, the term “food-grade” is meant to be understood as a material/substance/media that is safe for human consumption.

According to some embodiments, the above food-grade spirulina biomass is characterized by “L” coordinate in the range of 15<L<90 as measured according to CIELAB color space, wherein the maximum value (a+b) is equal to or smaller than (L*1.2). According to some embodiments, the above food-grade spirulina biomass is characterized by “L” coordinate of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 as measured according to CIELAB color space, wherein the maximum value (a+b) is equal to or smaller than (L*1.2).

According to some embodiments, the above food-grade spirulina biomass is characterized by “a” coordinate in the range of 0<a<95 as measured according to CIELAB color space, wherein the maximum value (a+b) is equal to or smaller than (L*1.2). According to some embodiments, the above food-grade spirulina biomass is characterized by “a” coordinate of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 92, 93, 94, 95 as measured according to CIELAB color space, wherein the maximum value (a+b) is equal to or smaller than (L*1.2).

According to some embodiments, the above food-grade spirulina biomass is characterized by “b” coordinate in the range of 0<b>55 as measured according to CIELAB color space, wherein the maximum value (a+b) is equal to or smaller than (L*1.2). According to some embodiments, the above food-grade spirulina biomass is characterized by is characterized by “b” coordinate of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55.

According to some embodiments, the above food-grade spirulina biomass is characterized by spectrum absorbance at the maximum absorbance peaks in the range of 400-560 nanometers.

According to some embodiments, the above food-grade spirulina biomass is characterized by spectrum absorbance of 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449,450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 92, 493, 494, 495, 496, 497, 498, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560.

According to some embodiments, the above food-grade spirulina biomass is characterized by RGB color space values in the range of 50>R>255 wherein each G and B is equal to or smaller than (R−30).

According to some embodiments, the above food-grade spirulina biomass is characterized by R value of RGB color space of 50, 55, 60, 65, 70, 75, 80, 85, 90, 95, 100, 105, 110, 115, 120, 125, 130, 135, 140, 155, 160, 165, 170, 175, 180, 185, 190, 200, 210, 215, 220, 225, 230, 235, 240, 245, 250, 255, wherein each G and B is equal to or smaller than (R−30).

An RGB color space is defined by chromaticity coordinates of the red, green, and blue additive primaries, the white point which is usually a standard illuminant, and the transfer function which is also known as the tone response curve (TRC) or gamma. Applying Grassmann's law of light additivity, a color space so defined can produce colors which are enclosed within the 2D triangle on the chromaticity diagram defined by those primary coordinates. The TRC and white point further define the possible colors, creating a volume in a 3D shape that never exceeds the triangular bounds.

According to some embodiments, the above food-grade spirulina biomass is characterized by CMYK color space values of C=0, 0.1>M>0.8, 0.1>Y>0.8, 0.01>K>0.5, wherein K<M*0.6 and M*1.5>Y>M*0.5.

The CMYK model works by partially or entirely masking colors on a lighter, usually white, background. Such a model is called subtractive because different materials “subtract” the colors red, green and blue from white light. White light minus red leaves cyan, white light minus green leaves magenta, and white light minus blue leaves yellow. In additive color models, such as RGB, white is the “additive” combination of all primary-colored lights, black is the absence of light. In the CMYK model, it is the opposite: white is the natural color of the paper or other background, black results from a full combination of colored materials.

According to some embodiments, the above food-grade spirulina biomass is characterized by having protein content in the range of 70% to 99% calculated based on dry weight.

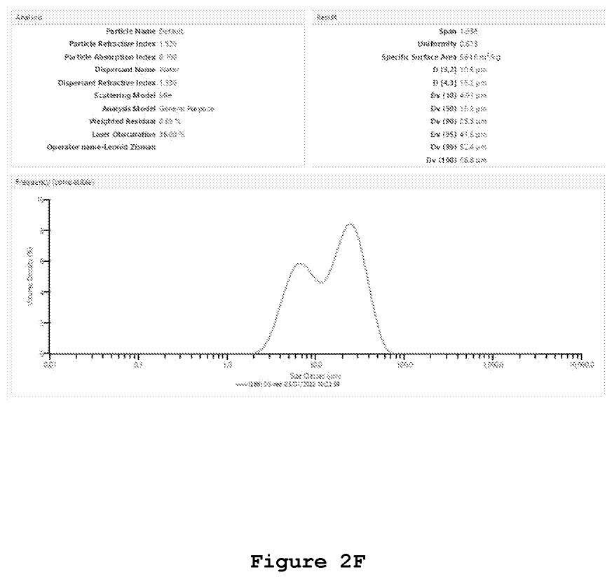

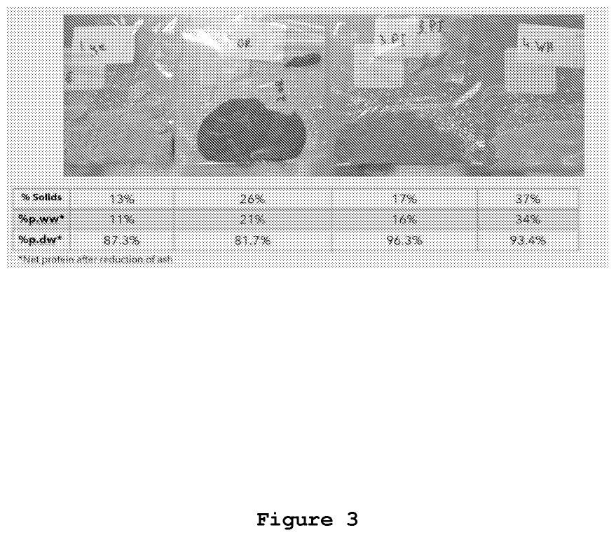

According to some embodiments, the above food-grade spirulina biomass is characterized by having protein content of 70%, 71%, 72%, 73%, 74%, 75%, 76%, 77%, 78%, 79%, 80%, 81%, 82%, 83%, 84%, 85%, 86%, 87%, 88%, 89%, 90%, 92%, 93%, 94%, 95%, 96%, 97%, 98%, 99% calculated based on dry weight. Reference is now made to FIG. 3, demonstrating protein content of spirulina biomass.

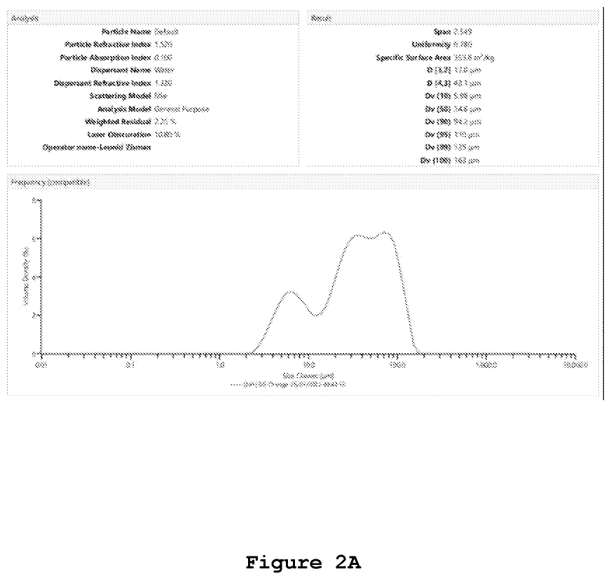

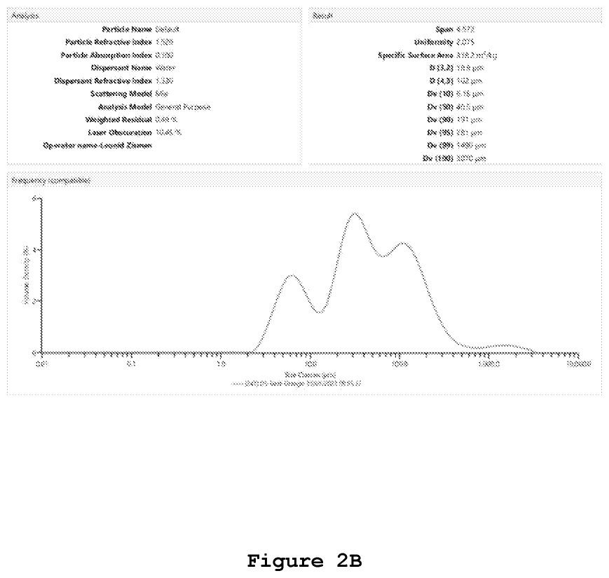

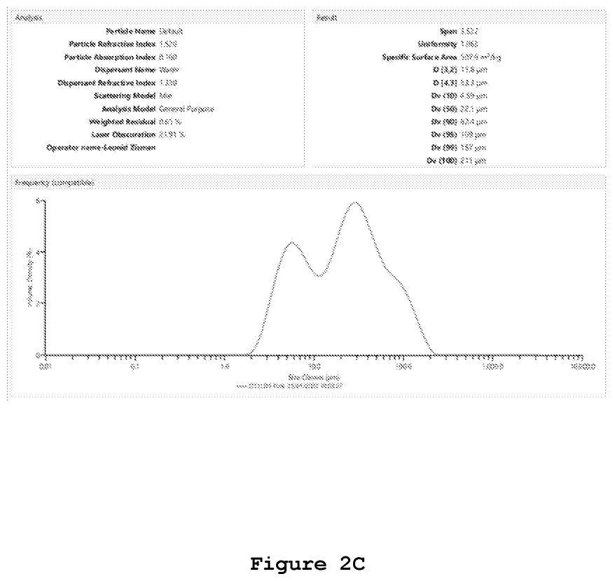

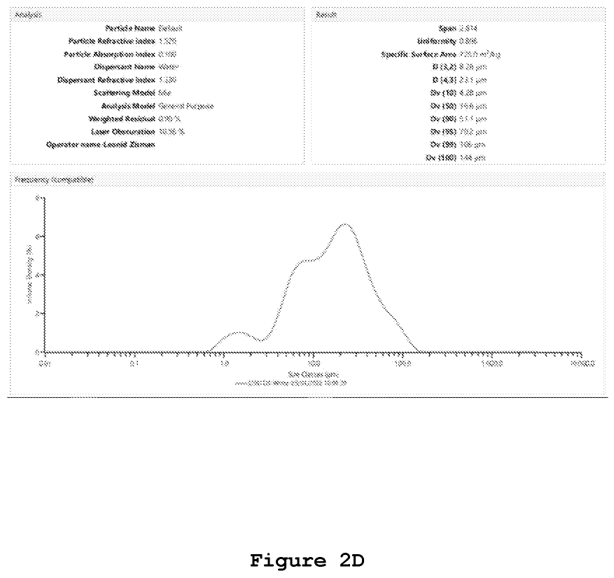

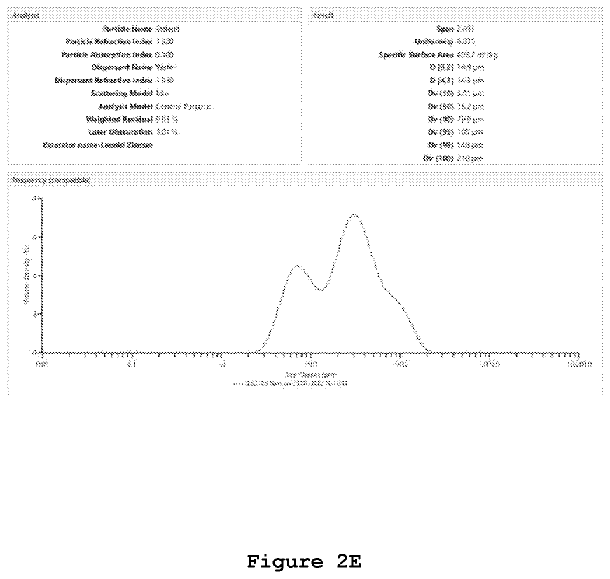

According to some embodiments, the above food-grade spirulina biomass is characterized by having a particle size in the range of 0.05 μm to 1000 μm. According to some embodiments, the particle size in the range of 0.05 μm to 100 μm. According to some embodiments, the particle size in the range 0.05 μm to 10 μm. According to some embodiments, the particle size in the range of 0.05 μm to 100 μm; 0.5 μm to 100 μm; 0.5 μm to 100 μm; 5 μm to 100 μm; 10 μm to 100 μm; 10 μm to 1000 μm; 100 μm to 1000 μm; μm; 10 μm to 500 μm; 50 μm to 500 μm; 0.05 μm to 500 μm; 0.5 μm to 500 μm; 0.05 μm to 300 μm; 0.05 μm to 200 μm; 200 μm to 1000 μm; 100 μm to 500 μm; 10 μm to 200 μm; 25 μm to 500 μm; 25 μm to 1000 μm. According to some embodiments, the above food-grade spirulina biomass is characterized by having a particle size of 1 μm>D(10)>8 μm. According to some embodiments, the above food-grade spirulina biomass is characterized by having a particle size of 10 μm>D(50)>80 μm. Reference is now made to FIG. 2A-F demonstrating particle size distribution of spirulina samples according to the color shade.

According to some embodiments, the invention provides an edible media prepared from and/or comprising the above food-grade biomass. As used herein, the term “edible” is meant to be understood, without limitation as safe and suitable for eating/consumption.

According to some embodiments, the above edible media is characterized by color coordinates in the range of 15<L<90, 0<a<95, 0<b<55, wherein the maximum value (a+b) is equal to or smaller than (L*1.2), as measured according to CIELAB color space.

According to some embodiments, the above edible media is characterized by “L” coordinate in the range of 15<L<90 as measured according to CIELAB color space, wherein the maximum value (a+b) is equal to or smaller than (L*1.2). According to some embodiments, the above edible media is characterized by “L” coordinate of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, as measured according to CIELAB color space, wherein the maximum value (a+b) is equal to or smaller than (L*1.2).

According to some embodiments, the above edible media is characterized by “a” coordinate in the range of 0<a<95 as measured according to CIELAB color space, wherein the maximum value (a+b) is equal to or smaller than (L*1.2). According to some embodiments, the above edible media is characterized by is characterized by “a” coordinate of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 92, 93, 94, 95, as measured according to CIELAB color space, wherein the maximum value (a+b) is equal to or smaller than (L*1.2).

According to some embodiments, the above edible media is characterized by “b” coordinate in the range of 0<b<55 as measured according to CIELAB color space, wherein the maximum value (a+b) is equal to or smaller than (L*1.2). According to some embodiments, the above edible media is characterized by is characterized by “b” coordinate of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, as measured according to CIELAB color space, wherein the maximum value (a+b) is equal to or smaller than (L*1.2).

According to some embodiments, the above edible media is in a substantially solid form, in a substantially liquid form, or in a semi solid form. In the context of the invention, the term “substantially solid” is meant to be understood as a state having identical, close, similar, or alike physical properties to the common solids. In the context of the invention, the term “substantially liquid” is meant to be understood as a state having identical, close, similar, or alike physical properties to the common liquid. As used herein, the term “semi-solid” refers, without limitation to a state that is in between a solid and a liquid. Another name for a semi-solid is a quasi-solid. At the microscopic scale, it has a disordered structure unlike the more common solids.

According to some embodiments, the above edible media is characterized by having a protein content in the range of 20% to 100% on a dry weight basis.

According to some embodiments, the above edible media is characterized by having protein content of 208, 218, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 60%, 62%, 63%, 64%, 65%, 66%, 67%, 68%, 69%, 70%, 71%, 72%, 73%, 74%, 75%, 76%, 778, 78%, 798, 80%, 81%, 82%, 83%, 84%, 85%, 86%, 87%, 88%, 89%, 90%, 92%, 93%, 94%, 95%, 96%, 97%, 98%, 99%, 100% as calculated based on dry weight.

According to some embodiments, the invention provides a food-grade bio-ink prepared from and/or comprising the above spirulina biomass, or the above edible media. In the context of the invention, the term “bio-ink” refers, without limitation, to materials used to produce engineered/artificial tissue and/or tissue-like structure using 3D printing. The bio-inks of the invention are composed of the cells originating from spirulina biomass in tandem with additional materials, such as, without limitation, biopolymers and/or any other components that may be beneficial for the performance of the bio-ink.

According to some embodiments, the above bio-ink further comprises, without limitation, at least one of: a binder, a cross-linker, a flavoring agent, a preservative, a thickener, an emulsifier, a colorant, and a food additive.

According to some embodiments, the invention provides a 3D printed food product prepared from the above bio-ink. In the context of the invention, 3D printed food product refers, without limitation, to a product manufactured by the process of 3D printing, specifically, 3D food printing. The 3D food printing is performed using food-grade bio-inks that may be customized for achieving specific shape, color, texture, flavor and/or nutrition values, and/or any other desired properties.

According to some embodiments, the above 3D printed food product is characterized by having a protein content in the range of 20% to 998. According to some embodiments, the above 3D printed food product is characterized by having protein content of 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 58%, 59%, 60%, 61%, 62%, 63%, 64%, 65%, 66%, 67%, 68%, 69%, 60%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 92%, 93%, 94%, 95%, 96%, 978, 98%, and 99%.

According to some embodiments, the above 3D printed food product is characterized by color coordinates in the range of 15<L<90, 0<a<95, 0<b<55, wherein the maximum value (a+b) is equal to or smaller than (L*1.2), as measured according to CIELAB color space. According to some embodiments the above 3D printed food product is characterized by “L” coordinate in the range of 15<L<90 as measured according to CIELAB color space, wherein the maximum value (a+b) is equal to or smaller than (L*1.2). According to some embodiments, the above 3D printed food product is characterized by “L” coordinate of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, as measured according to CIELAB color space, wherein the maximum value (a+b) is equal to or smaller than (L*1.2).

According to some embodiments, the above 3D printed food product is characterized by “a” coordinate in the range of 0<a<95 as measured according to CIELAB color space, wherein the maximum value (a+b) is equal to or smaller than (L*1.2). According to some embodiments, the above 3D printed food product is characterized by “a” coordinate of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 92, 93, 94, 95, as measured according to CIELAB color space, wherein the maximum value (a+b) is equal to or smaller than (L*1.2).

According to some embodiments, the above 3D printed food product is characterized by “b” coordinate in the range of 0 <b<55 as measured according to CIELAB color space, wherein the maximum value (a+b) is equal to or smaller than (L*1.2). According to some embodiments, the above 3D printed food product is characterized by “b” coordinate of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, as measured according to CIELAB color space, wherein the maximum value (a+b) is equal to or smaller than (L*1.2).

According to some embodiments, the invention provides a food product prepared from and/or comprising the above food grade spirulina biomass or the above edible media.

According to some embodiments, the above food product is characterized by having a protein content in the range of 20% to 99%. According to some embodiments, the above food product is characterized by having protein content of 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 60%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 92%, 93%, 94%, 95%, 96%, 97%, 98%, and 99%.

According to some embodiments, the above food product is characterized by color coordinates in the range of 15<L<90, 0<a<95, 0<b<55, wherein the maximum value (a+b) is equal to or smaller than (L*1.2), as measured according to CIELAB color space. According to some embodiments the above food product is characterized by “L” coordinate in the range of 15<L<90 as measured according to CIELAB color space, wherein the maximum value (a+b) is equal to or smaller than (L*1.2). According to some embodiments, the above food product is characterized by “L” coordinate of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, as measured according to CIELAB color space, wherein the maximum value (a+b) is equal to or smaller than (L*1.2).

According to some embodiments, the above food product is characterized by “a” coordinate in the range of 0<a<95 as measured according to CIELAB color space, wherein the maximum value (a+b) is equal to or smaller than (L*1.2). According to some embodiments, the above food product is characterized by “a” coordinate of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 92, 93, 94, 95, as measured according to CIELAB color space, wherein the maximum value (a+b) is equal to or smaller than (L*1.2).

According to some embodiments, the above food product is characterized by “b” coordinate in the range of 0<b<55 as measured according to CIELAB color space, wherein the maximum value (a+b) is equal to or smaller than (L*1.2). According to some embodiments, the above food product is characterized by “b” coordinate of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, as measured according to CIELAB color space, wherein the maximum value (a+b) is equal to or smaller than (L*1.2).

According to some embodiments, the above food product and/or 3D printed food product can be, without limitation, meat analogue, fish analogue, egg substitute, and dairy analogue. In the context of the invention the term “meat analogue” refers, without limitation, to a food industry term for a meat-like substance made from vegetarian and/or not-animal source ingredients. The term “meat analogue” can be replaced by any of the following terms: plant-based meat, vegan meat, meat substitute, mock meat, meat alternative, imitation meat, or vegetarian meat, fake meat, and/or faux meat. Meat analogues typically approximate certain features such as, without limitation, texture, flavor, appearance, and/or chemical characteristics of specific types of meat.

In the context of the invention the term “fish analogue” refers, without limitation, to a food industry term for an edible fish-like and/or seafood-like substance and/or product suitable for human consumption, which is of a non-animal origin. The term “fish analogue” can be replaced by any of the following terms: plant-based fish, vegan fish, fish substitute, mock fish, fish alternative, imitation fish, or vegetarian fish, fake fish or faux fish.

In the context of the invention the term “egg substitute” refers, without limitation, to food products which can be used to replace eggs in cooking and baking.

In the context of the invention the term “dairy analogue” refers, without limitation, to a food product of non-animal origin, either liquid, solid or semi-solid, that resembles the appearance, taste, texture, nutritional value, and/or any other feature of dairy products, such as, without limitation, cheese, milk, yogurt, cream, or any other diary like product, can be that consumed directly or, alternatively, can be used in cooking and/or baking. The term “dairy analogue” is interchangeable with any of dairy substitute, plant-based cheese and/or milk, vegan dairy and/or milk and/or cheese, milk and/or/cheese substitute, mock dairy and/or milk and/or cheese, dairy alternative, fake milk and/or cheese.

According to some embodiments, the above food product and/or 3D printed food product, is a ready-for-consumption product.

According to some embodiments, the above food product and/or 3D printed food product, require further preparation such as, without limitation, cooking, baking, seasoning, priming, marinating, preserving, freezing, heating, or any other action that might be required to transform the food product to a ready-for-consumption product.

According to some embodiments, the above food product and/or 3D printed food product might be, without limitation, syrup, beverage, jelly, bar, instant product, pudding, jam, paste, and a solid product.

According to some embodiments, the above food product and/or 3D printed food is vegetarian product and/or vegan product. As used herein, the term “vegan” refers, without limitation, to a product containing no animal ingredients or animal-derived ingredients. As used herein, the term “vegetarian product” refers, without limitation, to a product that meets vegetarian standards by not including meat and animal tissue products.

According to some embodiments, the invention provides a process for the preparation of spirulina-based biomass characterized by color coordinates in the range of 15<L<90, 0<a<95, 0<b<55, wherein the maximum value of (a+b) is equal to or smaller than (L*1.2), as measured according to CIELAB color space. The process comprises the steps of:
a. mixing Spirulina biomass with an organic solvent;
b. adding to the mixture an alkaline component to reach pH in the range of 8.0 to 14.0;
c. adding to the mixture a peroxide solution;
d. Homogenizing the mixture;
e. optionally, cooling the resulted mixture to the room temperature;
f. separating the biomass from the medium;
g. washing the biomass with an aqueous solution comprising a fresh polar organic solvent;
h. suspending the washed biomass in the water and reducing the pH to the range of 3.0 to 4.0; and,
i. separating the biomass and resuspending in water.

According to some embodiments of the above process, the non-limiting list of organic solvents includes acetone, ethyl acetate, hexane, heptane, dichloromethane, methanol, ethanol, polyethylene glycol, tetrahydrofuran, acetonitrile, dimethylformamide, toluene and dimethyl sulfoxide.

According to some embodiments of the above process, the alkaline component may be alkaline metal hydroxides or salts thereof, including without limitation, Potassium hydroxide, Potassium Carbonate, Potassium Hydro-carbonate, Sodium hydroxide, Sodium Carbonate, Sodium Hydro-carbonate, and Calcium Hydroxide.

According to some embodiments of the above process, the peroxide solution is an aqueous 1-50% peroxide solution. In one embodiment, the peroxide component is hydrogen peroxide or peracetic acid.

According to some embodiments of the above process, the mixture is homogenized inside a reaction chamber. In one embodiment, the mixture is stirred at 20° C.-60° C. for 0.5 to 5 hours.

According to some embodiments of the above process, the biomass is separated from the medium by at least one of gravitational settling, assisted sedimentation, filtration, flocculation, coagulation, centrifugation, dissolved gas floatation or any other technique, suitable for the separation of the biomass from the medium.

According to some embodiments of the above process, the concentration of the fresh polar organic solvent in the solution is in the range of 60% to 90%. A non-limiting list of polar organic solvent includes acetone, ethyl acetate, hexane, heptane, dichloromethane, methanol, ethanol, polyethylene glycol, tetrahydrofuran, acetonitrile, dimethylformamide, toluene and dimethyl sulfoxide.

According to some embodiments of the above process, the pH of the aqueous solution is reduced with ion exchange resins and/or food grade acids including, without limitation, Acetic acid, Phosphoric acid, Hydrochloric acid, and Citric acid.

According to some embodiments of the above process, the Spirulina biomass is separated from the aqueous solution via one of the commonly used solid-liquid separation methods including, without limitation, gravitational settling, assisted sedimentation, filtration, flocculation, coagulation, centrifugation, dissolved gas floatation or any other suitable technique.

The above spirulina biomass, edible media, food products and processes according to one or more of the above embodiments, each individually or all together might be used, without limitation, in any relevant field of food industry, including, without limitation, artificial meat industry, artificial fish industry, printed food products, or any other type of products and/or industries and/or enterprises that may benefit from the present invention and its embodiments.

### EXAMPLES

### Example 1: Preparation of Spirulina Based Samples

Spirulina biomass was mixed with ethanol; an alkaline component was added to the mixture to reach pH in the range of 8.0 to 14.0. A peroxide solution was further added and the mixture was homogenized. The resulted mixture was cooled to the room temperature and the biomass was separated from the medium. The biomass was washed with an aqueous solution comprising a fresh ethanol, suspended in the water and the pH was reduced to the range of 3.0 to 4.0. The biomass was then separated and re-suspended in the water.

### Example 2: Comparative Cie L*, a*, b* Coordinates of Food Products and Spirulina-Based Preparations

Spirulina samples were obtained using the process according to Example 1.

Food samples were purchased 2 hours prior to the experiment in a local supermarket.

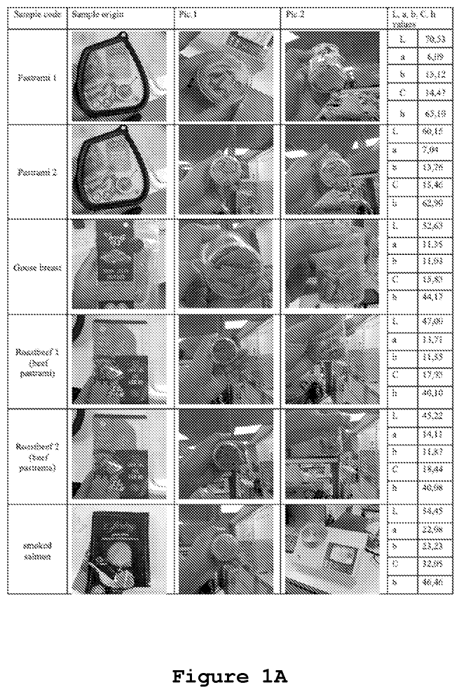

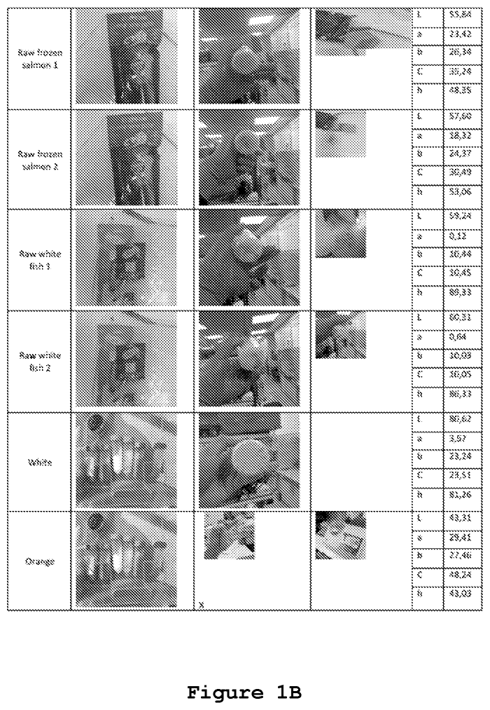

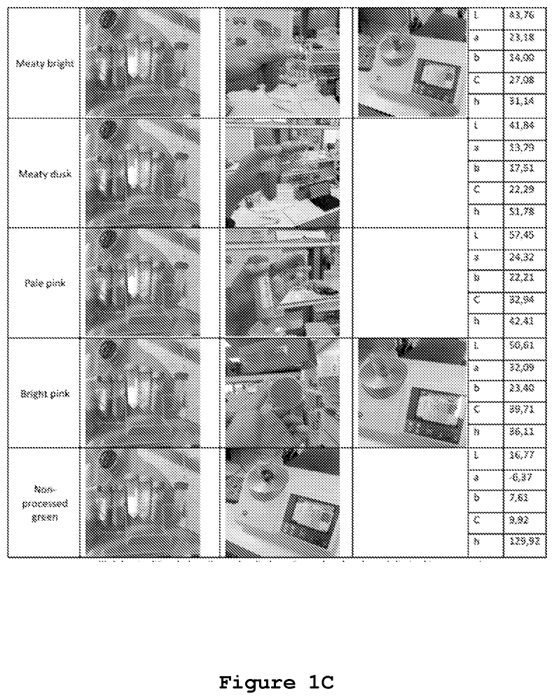

For color determination, the CR-5 colorimeter was calibrated on black and white standards on “Petri dish” mode. All measurements were taken in 100 ml glass. Food samples were cut into pieces, stirred with spatula and pressed to the bottom of the beaker. The experimental values and the corresponding visuals are presented in FIG. 1A-C.

The differences between different parts of the same samples were measured using the following formulas and are presented in Table 1:

$$Δ {E}_{\text{?}}^{\text{?}} = \sqrt{{( {L}_{2}^{\text{?}} - {L}_{1}^{\text{?}} )}^{2} + {( {a}_{2}^{\text{?}} - {a}_{1}^{\text{?}} )}^{2} + {( {b}_{2}^{\text{?}} - {b}_{1}^{\text{?}} )}^{2}}$$

$$Pic . 2 CIE 76 Δ E formula$$

$$Δ {E}_{\text{?}}^{\text{?}} = \sqrt{{( \frac{{L}_{2}^{\text{?}} - {L}_{1}^{\text{?}}}{{K}_{L}} )}^{\text{?}} + {( \frac{{C}_{2}^{\text{?}} - {C}_{1}^{\text{?}}}{1 \text{?} {K}_{1} {C}_{1}^{\text{?}}} )}^{\text{?}} + {( \frac{{\text{?}}_{\text{?}} - {\text{?}}_{\text{?}}}{1 \text{?} {K}_{2} {C}_{1}^{\text{?}}} )}^{\text{?}}}$$

$$Pic . 3 CIE 94 Δ E formula , {K}_{L} = 2 , {K}_{1} = 0.048 , {K}_{2} = 0.014$$

$$\text{?} \text{indicates text missing or illegible when filed}$$

According to the Commission internationale de l'éclairage (CIE), ΔE of average 2.3 is the least possible value of difference the human eye can observe.

TABLE 1
Evaluating ΔE max for CIE76 and CIE94 formulas

| | | | Raw salmon | Raw white fish |
|---|---|---|---|---|
| | Pastrami 1-2 | Roastbeef 1-2 | 1-2 | 1-2 |
| ΔE CIE76 | 10.44 | 1.85 | 5.74 | 1.26 |
| ΔE CIE94 | 5.35 | 0.93 | 3.43 | 1.48 |

It was decided to refer the values for Raw frozen salmon 1-2 as the ΔE max, providing the extreme color difference in the same exact sample. Therefore, if the difference between Spirulina and food samples would be less than ΔE max, it can be considered as similar to the original product. The color code represented in Table 2 was implemented. The value of 2*ΔE max was chosen to identify approximately similar results.

The results are presented in Table 3 and 4.

### Results and Discussion

The spirulina sample “Pale pink” is almost as close to raw salmon as another piece of salmon and is very similar to smoked salmon by color with the value 2.97 of ΔE CIE94 (3.43 for ΔE max). Some other samples, such as “Meaty dusk” and “Meaty bright” resemble meat products very close and “Bright pink” may be an essential artificial salmon component as well as “Orange”. Spirulina be useful for “White” may representation of fats, for example, salmon or meat, which seems to be more yellow.

### Conclusions

Examples from Spirulina color pallet were compared by color values with food products from a local store. It was found that the spirulina samples were very close or even identical to the color of smoked or raw salmon, as well as Meat analogues. Further investigation of bigger amounts of food and Spirulina materials is needed to achieve definite color ranges and differences.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. As used herein the terms “comprises”, “comprising”, “includes”, “including”, “having” and their conjugates mean “including but not limited to”. The term “consisting of” means “including and limited to”.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another component, region, layer and/or section.

It will be understood that when an element is referred to as being “on,” “attached” to, “operatively coupled” to, “operatively linked” “operatively to, engaged” with, “connected” to, “coupled” with, “contacting,” etc., another element, it can be directly on, attached to, connected to, operatively coupled to, operatively engaged with, coupled with and/or contacting the other element or intervening elements can also be present. In contrast, when an element is referred to as being “directly contacting” another element, there are no intervening elements present.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Whenever the term “about” is used, it is meant to refer to a measurable value such as an amount, a temporal duration, and the like, and is meant to encompass variations of ±20%, ±10%, ±5%, ±1%, or ±0.1% from the specified value, as such variations are appropriate to perform the disclosed methods.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

All publications, patent applications, patents, and other references mentioned in the disclosures of these publications in their entireties are hereby incorporated by reference into this application in order to more fully describe the state of the art to which this invention pertains. In case of conflict, the patent specification, including definitions, will prevail. In addition, the materials, methods, and examples are illustrative only and not intended to be limiting. Throughout this application various publications, published patent applications and published patents are referenced.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description. While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention. Various embodiments have been presented. Each of these embodiments may of course include features from other embodiments presented, and embodiments not specifically described may include various features described herein.

1. A food-grade spirulina biomass characterized by at least one of the following:
a) color coordinates in the range of 15<L<90, 0<a<95, 0<b<55, wherein the maximum value (a+b) is equal to or smaller than (L*1.2), as measured according to CIELAB color space;
b) having spectrum absorbance at the maximum absorbance peaks in the range of 400-560 nanometers;
c) RGB color space values in the range of 50>R>255 wherein each G and B is equal to or smaller than (R−30).
d) CMYK color space values of C=0, 0.1>M>0.8, 0.1>Y>0.8, 0.01>K>0.5, wherein K<M*0.6 and M*1.5>Y>M*0.5;
wherein the biomass has protein content in the range of 70% to 99% calculated based on dry weight.

2-5. (canceled)

6. The food-grade biomass of claim 1, characterized by having a particle size in the range of 0.05 μm to 1000 μm.

7. The food-grade biomass of claim 1, characterized by having a particle size of 1 μm>D(10)>8 μm.

8. The food-grade biomass of claim 1, characterized by having a particle size of 10 μm>D(50)>80 μm.

9. An edible media prepared from and/or comprising the food-grade biomass of claim 1.

10. The edible media of claim 9, characterized by color coordinates in the range of 15<L<90, 0<a<95, 0<b<55, wherein the maximum value (a+b) is equal to or smaller than (L*1.2), as measured according to CIELAB color space.

11. The edible media of claim 9, in a substantially solid form, in a substantially liquid form, or in a semi solid form.

12. The edible media of claim 9, having a protein content in the range of 20% to 100% on a dry weight basis.

13. A food-grade bio-ink prepared from and/or comprising the biomass of claim 1.

14. The food-grade bio-ink of claim 13, further comprising at least one of: a binder, a cross-linker, a flavoring agent, a preservative, a thickener, an emulsifier, a colorant, and a food additive.

15. A 3D printed food product prepared from the bio-ink of claim 13.

16. The 3D printed food product of claim 15, characterized by having a protein content in the range of 20% to 99%.

17. The 3D printed food product of claim 15, characterized by coordinates in the range of 15<L<90, 0<a<95, 0<b<55, wherein the maximum value (a+b) is equal to or smaller than (L*1.2), as measured according to CIELAB color space.

18. A food product prepared from and/or comprising the biomass of claim 1.

19. The food product of claim 18, characterized by coordinates in the range of 15<L<90, 0<a<95, 0<b<55, wherein the maximum value (a+b) is equal to or smaller than (L*1.2), as measured according to CIELAB color space at the room temperature.

20. The food product of claim 18, characterized by protein content of 20% to 100%.

21. The food product of claim 18, further comprising at least one of a flavoring agent, a food colorant, a binder, a preservative, a stabilizer, and a food additive.

22. The food product of claim 18, selected from the group consisting of meat analogue, fish analogue, egg substitute, and a dairy analogue.

23. A process for the preparation of spirulina-based biomass of claim 1, comprising the steps of:
a. mixing Spirulina biomass with an organic solvent;
b. adding to the mixture an alkaline component to reach pH in the range of 8.0 to 14.0;
c. adding to the mixture a peroxide solution;
d. Homogenizing the mixture;
e. optionally, cooling the resulted mixture to the room temperature;
f. separating the biomass from the medium;
g. washing the biomass with an aqueous solution comprising a fresh polar organic solvent;
h. suspending the washed biomass in the water and reducing the pH to the range of 3.0 to 4.0; and,
i. separating the biomass and resuspending in water.

24. A food product prepared from and/or comprising the edible media of claim 9.
